(19) United States
(12) Patent Application Publication
(10) Pub. No.: US 2019/0112128 A1
(43) Pub. Date: Apr. 18, 2019

(54) TRASH RECEPTACLE SYSTEM AND METHOD

(71) Applicant: Steve DeGeorge, Fountian Hills, AZ (US)

(72) Inventor: Steve DeGeorge, Fountian Hills, AZ (US)

(21) Appl. No.: 16/219,670

(22) Filed: Dec. 13, 2018

Related U.S. Application Data
(63) Continuation-in-part of application No. 15/818,293, filed on Nov. 20, 2017.
(60) Provisional application No. 62/459,056, filed on Feb. 14, 2017.

(51) Int. Cl. B65F 7/00; A01M 1/20

(52) U.S. Cl. CPC B65F 7/00; B65F 2210/139; B65F 2210/129; A01M 1/2044

(57) ABSTRACT

A receptacle which includes a body, a lid, and a deodorizing and sanitizing mechanism. The body may have an open upper region, a lower region, and an internal cavity, with the lid being pivotally coupled to a first portion of the open upper region of the body, the lid configured to close the open upper region of the body, and the deodorizing and sanitizing mechanism being removably attached or built within body to a second portion of the open upper region of the body. The deodorizing and sanitizing mechanism is configured to lessen malodorous and unsanitary conditions inside the receptacle. The receptacle further including an illumination device that is designed to provide light to the receptacle. The trash receptacle system is useful for deodorizing and sanitizing a trash receptacle.

### CROSS-REFERENCE TO RELATED APPLICATION(S)

The present application is a Continuation-in-Part application related to and claims priority to Pending U.S. Non-Provisional application Ser. No. 15/818,293 filed Nov. 20, 2017 and U.S. Provisional Patent Application No. 62/459,056 filed Feb. 14, 2017, which are incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of sanitation of existing art and more specifically relates to odor, sanitizing and pest management.

### RELATED ART

Many homes have garbage cans. As garbage cans fill up with food scraps, discarded containers, and other trash, not only can the smell become pungent and unpleasant but it can also waft out into a room. Therefore, many people have to take out the trash before the bag is entirely full, which can be wasteful. Additionally, pesky flies, mosquitoes and insects can hover around the trash or even get inside and lay eggs. An efficient alternative is desired.

U.S. Pat. No. 7,878,359 to Wen-Hsiung Ko relates to a trash can with a deodorizing mechanism. The described trash can with a deodorizing mechanism includes a trash can comprising a body having an opening formed at the top of the body, a lid pivotally coupled to the top of the body for performing an opening or closing movement, and a deodorizing mechanism installed on the body and including a bottle and a nozzle. The nozzle includes an actuating element propped by an elastic body to provide a restoring elasticity, such that if the lid is closed and covered onto the opening, the actuating element will be pressed by the lid to compress the elastic body, and the nozzle will suck a deodorant stored in the bottle and spray the deodorant towards the interior of the body. If the lid is closed and covered onto the opening, the elastic body will be compressed to slow down the closing movement of the lid.

### SUMMARY OF THE INVENTION

In view of the foregoing disadvantages inherent in the known odor management art, the present disclosure provides a novel trash receptacle system. The general purpose of the present disclosure, which will be described subsequently in greater detail, is to provide an efficient and effective trash receptacle system.

A receptacle is disclosed herein. The receptacle includes a body, a lid, and a deodorizing, pest control and sanitizing mechanism. The body may have an open upper region, a lower region, and an internal cavity, with the lid being pivotally coupled to a first portion of the open upper region of the body, the lid configured to close the open upper region of the body, and the deodorizing and sanitizing mechanism being removably attached to a second portion of the open upper region of the body. The deodorizing and sanitizing mechanism is configured to lessen (minimize) malodorous and unsanitary conditions inside the receptacle.

The deodorizing and sanitizing mechanism includes a container having an actuating element with an elastic body, configured to provide elasticity to the actuating element when the lid touches the actuating element. The deodorizing and sanitizing mechanism further comprises a heavy spring coupled to the actuating element, and a planar top piece. The planar top piece is coupled to the heavy spring so that the lid is configured to touch the planar top piece and actuate the release of a deodorizing and sanitizing compound.

The planar top piece actuates a button. The button may be configured to actuate an illumination device that is designed to provide light to the receptacle. The button is coupled to a body, and configured to actuate the deodorizing and sanitizing mechanism. The deodorizing and sanitizing mechanism actuating element is a nozzle attached to a container which is a bottle. The bottle may be replaceable so that new deodorizer and sanitizer may be inserted into a removable angled bracket or molded into the top upper region of the body. The removable-or molded angled bracket attaches the deodorizing and sanitizing mechanism to the open upper region of the body. In further embodiments, the angled bracket may allow for the deodorizing and sanitizing mechanism to be used with various other waste receptacles. The deodorizing and sanitizing mechanism may be formed from the group consisting of a deodorizer, a pesticide, a sanitizer, and a compost accelerate. In some embodiments, the illumination device interacts with an automatic timer to turn the illumination on or off. In these or other embodiments, the illumination device or the deodorizing and sanitizing mechanism interacts with an light sensor such as an ambient light sensor so that the device or illumination only operate in the evening or after dark.

According to another embodiment, a method of deodorizing and sanitizing a receptacle is also disclosed herein. The method of deodorizing and sanitizing a receptacle includes closing a lid of a receptacle, allowing for the lid to touch a deodorizing and sanitizing mechanism, and actuate the release of a deodorizing and sanitizing compound. Alternately, manually pressing the deodorizing and sanitizing mechanism, and actuating the release of a deodorizing and sanitizing compound. In certain embodiments the spray bottle may be attached directly to the side of the container using VELCRO/two-sided tape.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a trash receptacle system, constructed and operative according to the teachings of the present disclosure.

FIG. 1 is a perspective view of the receptacle during an ‘in-use’ condition, according to an embodiment of the disclosure.

FIG. 2 is a perspective view of the receptacle of FIG. 1, according to an embodiment of the present disclosure.

FIG. 3 is an inside perspective view of the receptacle of FIG. 1, according to an embodiment of the present disclosure.

FIG. 4 is a perspective view of the receptacle of FIG. 1, according to an embodiment of the present disclosure.

FIG. 5 is a flow diagram illustrating a method of (use for) deodorizing and sanitizing a receptacle, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to odor management and more particularly to a trash receptacle system as used to improve the conditions of the inside of a trash can to reduce odor and insect activity.

Generally, homes use trash bags inside trash cans to contain trash or garbage by lining the inside of the can. Trash bags help with messy, wet trash and garbage, however there is need to deal with the ‘smell’ that comes from the trash can. The present trash receptacle system and method provides a means to combat malodorous garbage. In a preferred embodiment, the system includes a trash receptacle having a deodorizing and sanitizing mechanism. In this embodiment, the deodorizing and sanitizing mechanism is separable from the trash receptacle. The deodorizing and sanitizing mechanism may include a means to attach the mechanism to a trash receptacle, allowing the mechanism to be used with a wide variety of trash receptacles. In operation, the mechanism may be configured to disperse a solution to the interior of the trash receptacle. The mechanism my further include an illumination device configured to activate upon opening the lid of a trash receptacle.

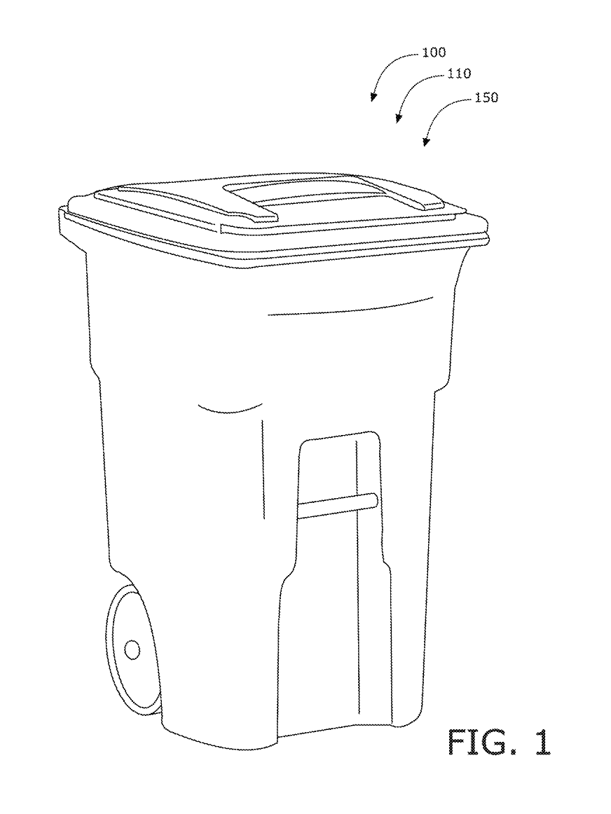

Referring now more specifically to the drawings by numerals of reference, there is shown in FIGS. 1-4, various views of a receptacle 100.

FIG. 1 shows a receptacle 100 during an ‘in-use’ condition 150, according to an embodiment of the present disclosure. Here, the receptacle 100 may be beneficial for use by a user 40 to reduce odor and insect activity inside a trash receptacle. As illustrated, the receptacle 100 may include a body 110, a lid 112, and a deodorizing and sanitizing mechanism 114. The body 110 may have an open upper region 116, a lower region 118, and an internal cavity 120. The lid 112 may be pivotally coupled to a first portion of the open upper region 116 of the body 110. The lid 112 may further be configured to close the open upper region 116 of the body 110. The deodorizing and sanitizing mechanism 114 may be attached within the open upper region 116 of the body 110, and may even further be configured to lessen malodorous and unsanitary conditions inside the receptacle 100.

According to one embodiment, the receptacle 100 may be arranged as a kit 105. In particular, the receptacle 100 may further include a set of instructions 107. The instructions 107 may detail functional relationships in relation to the structure of the receptacle 100 such that the receptacle 100 can be used, maintained, or the like, in a preferred manner.

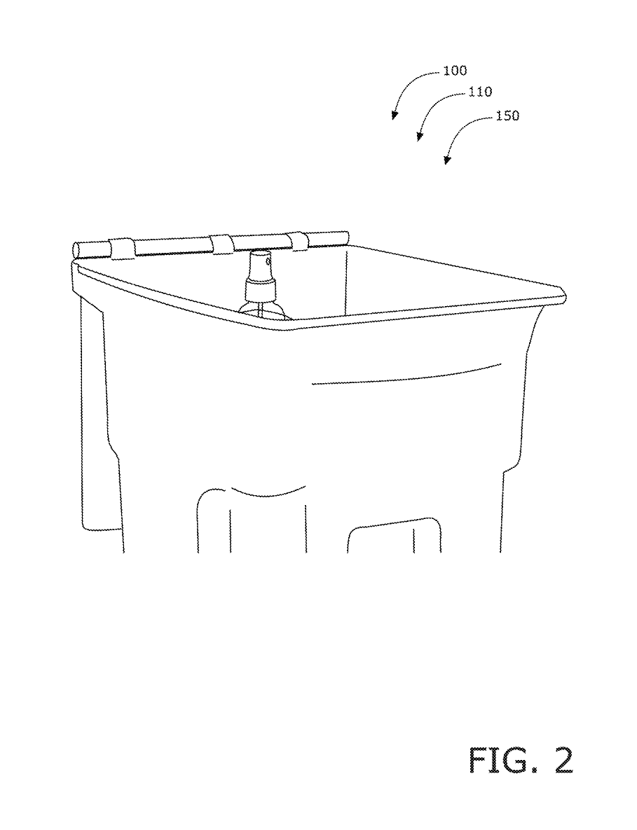

FIG. 2 shows the receptacle 100 of FIG. 1, according to an embodiment of the present disclosure. As above, the receptacle 100 may include a body 110, a lid 112, and a deodorizing and sanitizing mechanism 114. The body 110 may include an open upper region 116, a lower region 118, and an internal cavity 120.

The deodorizing and sanitizing mechanism 114 may include a container 122 including an actuating element 124. The actuating element 124 may be an elastic body configured to provide elasticity to the actuating element 124 when the lid 112 touches the actuating element 124. The deodorizing and sanitizing mechanism 114 may further include heavy spring 126 coupled to the actuating element 124, and a planar top piece 128. The lid 112 may be configured to actuate the deodorizing and sanitizing mechanism 114 by depressing the planar top piece 128 onto the actuating element 124, thereby releasing a deodorizing and sanitizing compound 130.

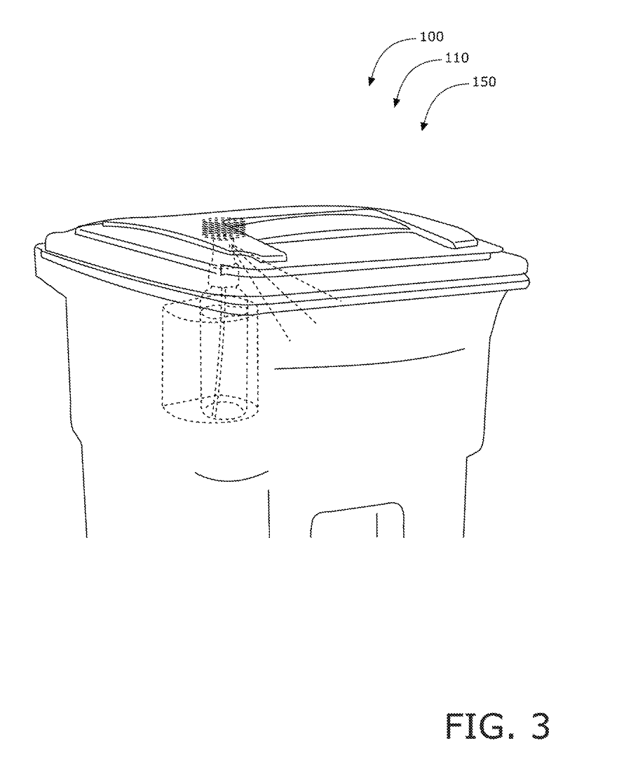

FIG. 3 is an inside perspective view of the receptacle 100 of FIG. 1, according to an embodiment of the present disclosure. The actuating element 124 may be a nozzle attached to a container 122 which may be a bottle. The bottle may be replaceable once the deodorizing and sanitizing compound 130 has been expended. The open upper region 116 of the body 110 may contain a removable angled bracket 132. The removable angled bracket 132 may hold the bottle containing the deodorizing and sanitizing compound 130. Alternately, the removable angled bracket 132 may be integral to the body. The deodorizing and sanitizing compound 130 may be a combination of a deodorizer, a pesticide, a sanitizer, and/or a compost accelerate.

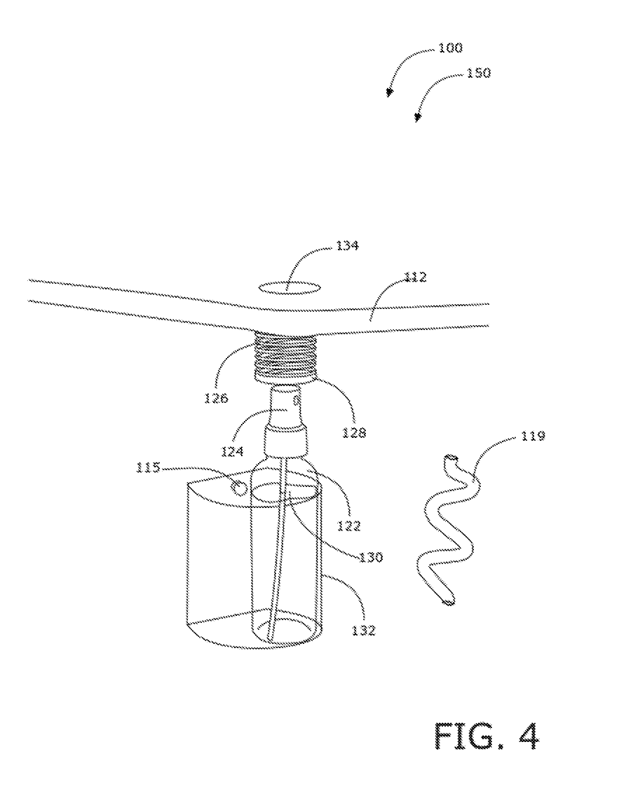

FIG. 4 is a perspective view of the receptacle of FIG. 1, according to an embodiment of the present disclosure. The lid 112 (FIG. 3) of the receptacle 100 is closed so that the deodorizing and sanitizing compound 130 may be disbursed within the internal cavity 120 of the body 110. In a preferred embodiment, the deodorizing and sanitizing mechanism 114 may include an illumination device 115. The illumination device 115 may be configured to illuminate the internal cavity 120 (FIG. 2) of the receptacle 100 (FIG. 3). In one embodiment, the illumination device 115 may produce ultraviolet light configured to kill bacteria. The illumination device 115 may be actuated by the lid 112. The lid 112 may decompress the heavy spring 126, thereby actuating the illumination device 115. Further means to activate the illumination device 115 may include, a photosensor (not shown), a trigger (not shown), and further conceived means of actuating the illumination device 115 to illuminate the internal cavity 120 of the receptacle 100. A hose 119 may further be included with the receptacle 100. The hose 119 may be useful for enabling the deodorizing and sanitizing mechanism 114 to farther dispose the deodorizing and sanitizing compound 130 (FIG. 3).

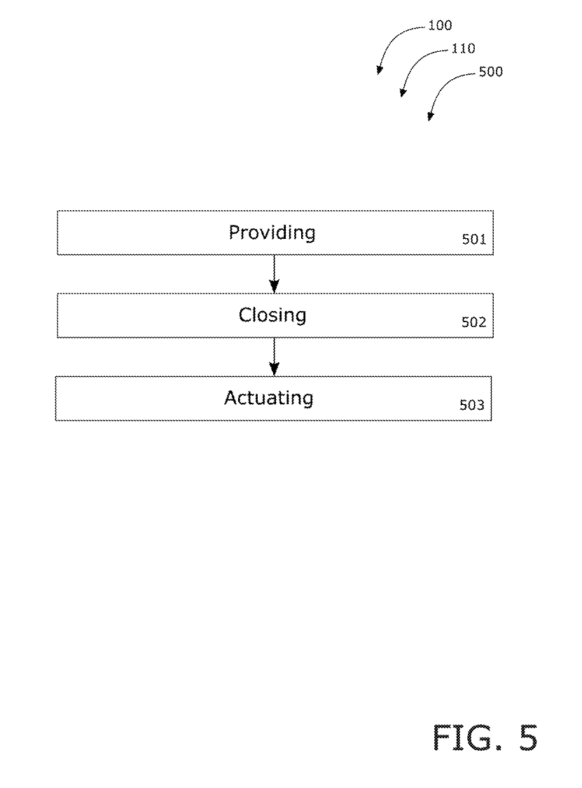

FIG. 5 is a flow diagram illustrating a method for deodorizing and sanitizing a receptacle, according to an embodiment of the present disclosure. In particular, the method for deodorizing and sanitizing a receptacle 500 may include one or more components or features of the receptacle 100 as described above. As illustrated, the method for deodorizing and sanitizing a receptacle 500 may include the steps of: step one 501, providing a receptacle 100 that houses one or more items; step two 502, closing a lid 112 of a receptacle 100; step three 503, actuating a deodorizing and sanitizing mechanism 114; and step four 504, pressing a button 134 on the outside of the receptacle 100 to actuate the deodorizing and sanitizing mechanism 114.

The receptacle 100 may comprise a body 110, the body 110 having an open upper region 116, a lower region 118, and an internal cavity 120; a lid 112, the lid 112 pivotally coupled to a first portion of an open upper region 116 of the body 110, with the lid 112 configured to close the internal cavity 120; a deodorizing and sanitizing mechanism 114 removably attached to a second portion of the open upper region 116 of the body 110. Closing the lid 112 allows the lid 112 to touch the deodorizing and sanitizing mechanism 114, consequently releasing a deodorizing and sanitizing compound 130 within the trash receptacle system. Alternately, a button 134 may be pressed on the outside of the receptacle 100 to release a deodorizing and sanitizing compound 130 within the trash receptacle system.

It should be noted that step 504 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for deodorizing and sanitizing a receptacle, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A receptacle for housing one or more items, said receptacle comprising;
a body, said body having an open upper region, a lower region, and an internal cavity;
a lid, said lid being pivotally coupled to a first portion of said open upper region of said body, said lid configured to close said open upper region of said body;
a deodorizing and sanitizing mechanism, said deodorizing and sanitizing mechanism being removably attached to a second portion of said open upper region of said body; and
wherein said deodorizing and sanitizing mechanism is configured to lessen malodorous and unsanitary conditions inside said receptacle.

2. The receptacle of claim 1, wherein said deodorizing and sanitizing mechanism includes a container, said container having an actuating element.

3. The receptacle of claim 2, wherein said actuating element is an elastic body, said elastic body configured to provide elasticity to the actuating element when said lid touches said actuating element.

4. The receptacle of claim 2, wherein said deodorizing and sanitizing mechanism further comprises heavy spring coupled to the actuating element, and a planar top piece, said planar top piece coupled to said heavy spring.

5. The receptacle of claim 4, wherein said lid is configured to touch said planar top piece and actuate release of a deodorizing and sanitizing compound.

6. The receptacle of claim 1, further comprising a button, said button coupled to said body and configured to actuate said deodorizing and sanitizing mechanism.

7. The receptacle of claim 3, wherein said actuating element is a nozzle.

8. The receptacle of claim 2, wherein said container is a bottle.

9. The receptacle of claim 8, wherein said bottle is replaceable.

10. The receptacle of claim 1, further comprising a removable angled bracket, said removable angled bracket is configured to attach the deodorizing and sanitizing mechanism to said second portion of said open upper region of said body.

11. The receptacle of claim 5, wherein the deodorizing and sanitizing compound is selected from the group consisting of a deodorizer, a pesticide, a sanitizer, and a compost accelerate.

12. The receptacle of claim 1, further comprising an angled bracket, wherein said angled bracket is an integral part of said body.

13. The receptacle of claim 1, wherein said deodorizing and sanitizing mechanism is also a pest control mechanism.

14. The receptacle of claim 2, wherein said deodorizing and sanitizing mechanism includes an illumination device.

15. The receptacle of claim 2, wherein said deodorizing and sanitizing mechanism includes a hose.

16. A receptacle for housing one or more items, said receptacle comprising;
a body, said body having an open upper region, a lower region, and an internal cavity;
a lid, said lid being pivotally coupled to a first portion of said open upper region of said body,
said lid configured to close said open upper region of said body;
a deodorizing and sanitizing mechanism, said deodorizing and sanitizing mechanism being
removably attached to a second portion of said open upper region of said body;
wherein said deodorizing and sanitizing mechanism is configured to lessen malodorous and
unsanitary conditions inside said receptacle;
wherein said deodorizing and sanitizing mechanism includes a container, said container having an actuating element;
wherein said actuating element is an elastic body, said elastic body configured to provide elasticity to the actuating element when said lid touches said actuating element;
wherein said deodorizing and sanitizing mechanism further comprises heavy spring coupled to the actuating element, and a planar top piece, said planar top piece coupled to said heavy spring;
wherein said lid is configured to touch said planar top piece and actuate release of a deodorizing and sanitizing compound;
further comprising a button, said button coupled to said body and configured to actuate said deodorizing and sanitizing mechanism;
wherein said actuating element is a nozzle;
wherein said container is a bottle;
wherein said bottle is replaceable;
further comprising a removable angled bracket, said removable angled bracket configured to attach the deodorizing and sanitizing mechanism to said second portion of said open upper region of said body;
wherein said deodorizing and sanitizing mechanism includes an illumination device;
wherein said deodorizing and sanitizing mechanism includes a hose; and
wherein the deodorizing and sanitizing compound is selected from the group comprising of a deodorizer, a pesticide, a sanitizer, and a compost accelerate.

17. A method of deodorizing and sanitizing a receptacle, said method comprising the steps of:
providing a receptacle that houses one or more items, said receptacle comprising a body, said body having an open upper region, a lower region, and an internal cavity; a lid, said lid being pivotally coupled to a first portion of said open upper region of said body, said lid configured to close said open upper region of said body; a deodorizing and sanitizing mechanism, said deodorizing and sanitizing mechanism being removably attached to a second portion of said open upper region of said body; and
closing said lid of said receptacle, wherein closing said lid allows for said lid to touch said deodorizing and sanitizing mechanism and actuate the release of a deodorizing and sanitizing compound for lessening malodorous and unsanitary conditions inside of said receptacle.

18. The method of claim 17, further comprising the step of:
pressing a button, said button coupled to said body and configured to actuate said deodorizing and sanitizing mechanism.
